(19) United States
(12) Patent Application Publication
(10) Pub. No.: US 2021/0038082 A1
(43) Pub. Date: Feb. 11, 2021

(54) HANDHELD PET DIAGNOSTIC DEVICE AND METHODS OF USE

(71) Applicant: Deanna Lynn Vicera, Bayville, NJ (US)

(72) Inventor: Deanna Lynn Vicera, Bayville, NJ (US)

(21) Appl. No.: 16/534,782

(22) Filed: Aug. 7, 2019

(51) Int. Cl. A61B 5/00; G16H 30/40

(52) U.S. Cl. CPC A61B 5/0077; A61B 5/0082; A61B 2503/40; A61B 5/0013; G16H 30/40

(57) ABSTRACT

A handheld diagnostic device includes a housing having a proximal end and a distal end, a nozzle, a magnifying element, at least one light, and a flow control configured and arranged to adjust a flow rate of a gas flowing through the nozzle.

### FIELD OF THE DISCLOSURE

The present disclosure relates to devices and methods for treating animals. More particularly the present disclosure relates to a handheld device to aid in diagnosing and treating pets and animals.

### BACKGROUND OF THE DISCLOSURE

Pets, zoo animals, and other wildlife may suffer from skin conditions, infections, parasites such as ticks and fleas, insect bites, dog or animal bites, or various other illnesses or diseases such as cancerous masses such as basil or squamous cell carcinomas, lesions, warts, moles, blood disorders, thyroid conditions or other disorders that can be seen on, or change the appearance of the skin. Typically, owners of such pets may be unaware of the ailment, or may notice unusual behavior of the pet without knowing the cause. Early detection is a key component to diagnosing a pet or species of wildlife for determining the proper course of treatment giving the pet or animal the best prognosis for recovery, reducing their unknown suffering, and possibly giving them an extension on their life span. One of the best ways to diagnose such ailments is through visual inspection, but this may be difficult because of the animal's coat of fur and especially if the animal is double coated or has a thick undercoat. To properly inspect such animals requires the pet owner or veterinarian to manually separate strands of fur with their fingers and look at the animal's skin. This can be both time-consuming and ineffective. Often, it may be difficult to see the skin of the animal without shaving the animal. Additionally, it may require touching possibly infected areas, posing a health risk to the owner and or veterinarian.

Thus, it would be in many of the above stated ways beneficial to have a handheld device to aid in inspecting a pet or animal and diagnosing various ailments.

### SUMMARY OF THE DISCLOSURE

In some embodiments, a handheld diagnostic device includes a housing having a proximal end and a distal end, a nozzle, at least one light, and a flow control configured and arranged to adjust a flow rate of a gas flowing through the nozzle.

In some embodiments, a method of examining an animal includes providing a handheld diagnostic device including a housing having a proximal end and a distal end, a nozzle, at least one light, and a flow control configured and arranged to adjust a flow rate of a gas flowing through the nozzle, and directing a stream of gas out of the nozzle onto a target area of the animal to blow the fur.

### BRIEF DESCRIPTION OF THE DISCLOSURE

Various embodiments of the presently disclosed handheld device are disclosed herein with reference to the drawings, wherein:

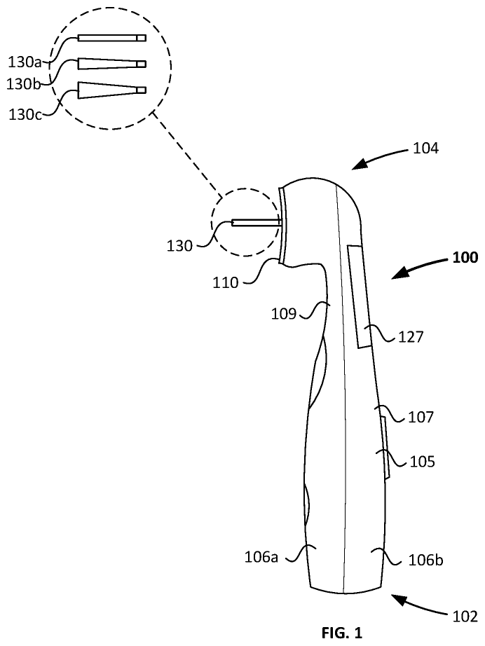

FIG. 1 is a schematic side view of a diagnostic device according to one embodiment;

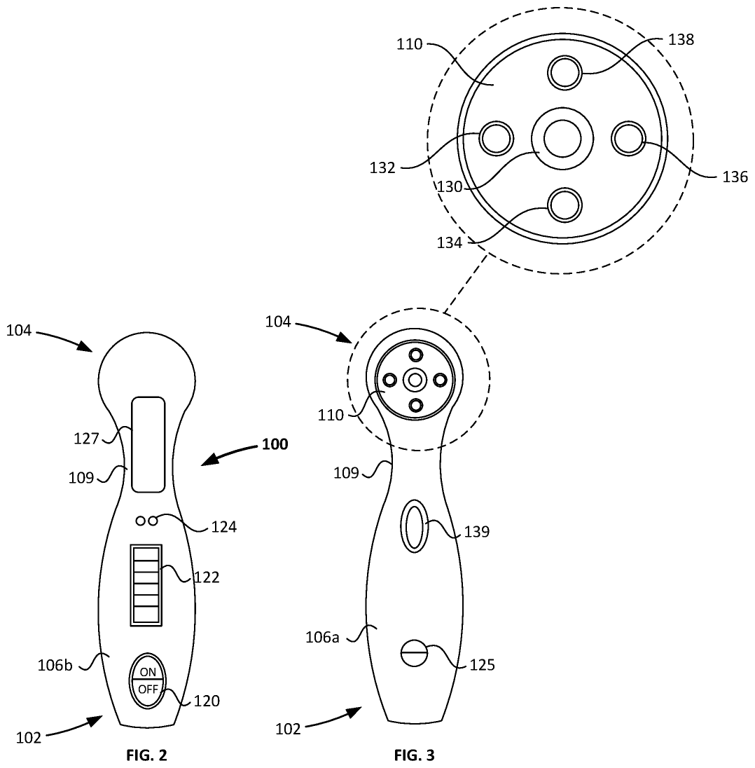

FIGS. 2 and 3 are schematic back and front views of the diagnostic device of FIG. 1;

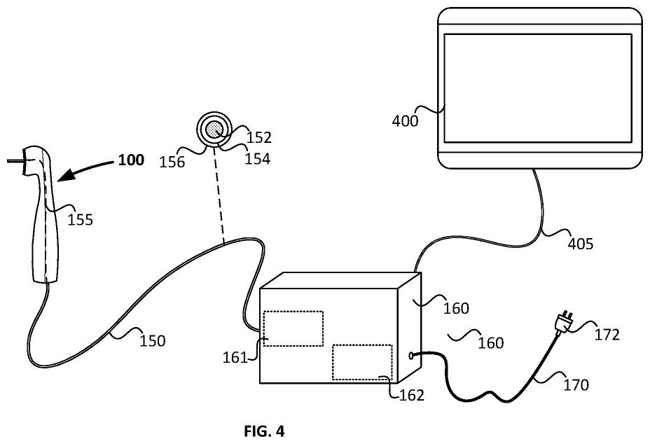

FIG. 4 is a schematic representation showing the diagnostic device of FIG. 1 in use with a base as part of a system; and

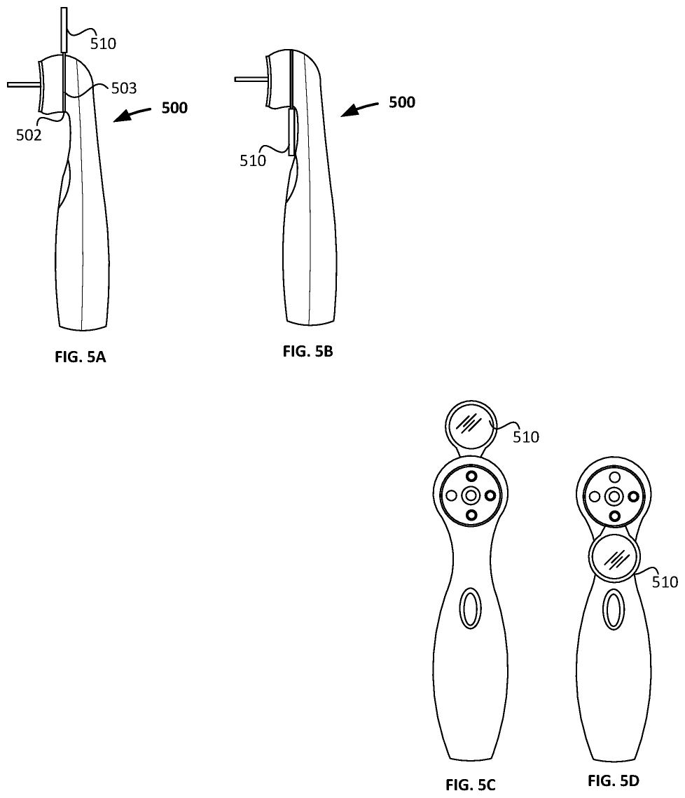

FIGS. 5A-D are schematic side and front representations showing another example of a diagnostic device having a physical magnifying lens.

Various embodiments of the present invention will now be described with reference to the appended drawings. It is to be appreciated that these drawings depict only some embodiments of the invention and are therefore not to be considered limiting of its scope.

### DETAILED DESCRIPTION

Despite the various Improvements that have been made to pet and animal care, conventional devices and methods suffer from certain shortcomings such as those discussed above.

There therefore is a need for further improvements to the devices and methods used to diagnose and care for pets. Among other advantages, the present disclosure may address one or more of these needs.

FIG. 1 illustrates one example of a handheld diagnostic tool 100 to improve care of pets and animals. Handheld tool 100 includes a plastic, resin or metallic housing 105 and generally extends between a proximal end 102 and a distal end 104. Housing 105 may be divided into a front cover 106a and a rear cover 106b, and may include various sections such as a gripping body 107, a neck 109 and a face 110. As shown, face 110 includes an air nozzle 130 having an outer diameter of between ⅛ and 1.5 inches, the nozzle being capable of directing air at a flow rate that is sufficient to part the fur or coat of the animal from a distance of 3 inches to 24 inches from the animal. In some example, the nozzles may be replaceable, and the user may select any of nozzles 130a, 130b, 130c, each having various shapes (e.g., cylindrical or conical) and diameters so that the user is able to choose a wider stream of air (e.g., nozzle 130c) or a more focused stream of air (e.g., nozzle 130a).

Turning to FIG. 2, rear cover 106b may have an on/off switch 120 adjacent the proximal end 102, and a flow control 122 to adjust the flow rate of air, the flow control 122 being located approximately halfway between the proximal and distal ends. In some examples, flow control 122 may be in the form of a wheel that is capable of rotating upward to increase the flow rate, or downward to decrease the flow rate of air. In other examples, the flow control is digital and the user may press a button to adjust the flow rate. In some examples, the flow rate of air may be chosen between a number of settings (e.g., low, medium, high) as determined by the type of coat. A series of buttons 124 may also be used to control the camera and/or lights described below. A zoom toggle 125 may include two buttons to zoom in and out of the target area that is being recorded as will be described in greater detail below. An optional display 127 (e.g., LCD display) may also be disposed on the rear cover 106b to allow a user to see operation parameters such as flow rate, battery life, status, wireless or wired connectivity status as well as other suitable parameters. LCD display may also have a series of inputs or may be a touchscreen that is used to control the parameters of the device (e.g., zoom, image capture, flow rate, lights, etc.). It will be understood that though certain buttons, toggles and controls are described as being on the front or rear cover, that the location of the buttons, toggles and controls may be altered as desired.

The front of the device (FIG. 3) may include a circular face 110 as previously noted, and the face may include a number of instruments for air delivery, visualization and/or data capture. In at least some examples, in addition to air nozzle 130, face 110 may include a number of instruments disposed around the nozzle including a lighting element 132 (e.g., an LED light) to illuminate the body of the animal to be inspected, an ultraviolet light 134, a camera 136, and a laser 138. Camera 136 may be capable of capturing an image or video of a target area, and may include zoom in/out features controlled by zoom toggle 125 to magnify the field of view. An image capture button 139 may be disposed on the front cover to take photographs or videos by either pressing the button once, or by holding the button to take a video.

Diagnostic tool 100 may be coupled at proximal end 102 to a flexible tubing 150 that includes an air delivery lumen 152, a power cable 152 and a communication wire 154 for transmitting information from the tool to a base 160. The flexible tubing 160 may be coupled at the other end to a base 160 that serves as both an air compressor 161 and a data storage unit 162. An internal conduit 155 extends from the flexible tubing 150 and the nozzle and is in communication with the nozzle and the air delivery lumen 152. In one example, base 160 may include a processor and memory (not shown) to store visual information captured from the camera. Base 160 may also be connected via wireless means (e.g., WIFI) or via wired means (e.g., HDMI cable 405) to the internet to transmit data, information, or an image to another a display 400, a computer or a mobile device (e.g., smartphone). Alternatively, handheld diagnostic tool itself may directly connect through wireless or wired means to a computer, smartphone or tablet. For example, display 400 may show in real-time, the target area that is being captured by the camera of diagnostic tool 100, and the user may use zoom toggle to magnify this area. Base 160 may include an electrical power cord 170 and a plug 172 for coupling with an electrical outlet. Alternatively, base 160 may be battery or solar-powered. Base 160 may also include sound-proofing features (e.g., be made out of a suitable polymeric material, or include other noise-reducing or noise-cancelling features) to reduce the noise from the compressor. Base 160 may also include an opening for mating with a conduit that couples to a tank or air reservoir.

In use, handheld tool 100 may be used by a veterinarian or technician in an office setting, or by a pet owner at home to inspect an animal. Such inspection may be done on a regular basis as part of a wellness routine, or as necessary if the pet owner or veterinarian notices symptoms or behavior signifying an ailment, or if the pet has been involved in a violent incident with another animal. The user may grip the handheld tool upright as shown in FIG. 1 with face 110 being directed toward the target area of the animal. By pressing the on/off switch 120, the user may turn on the device, and with it the compressor 161 within base 160. An air stream will begin to flow through the air lumen into the handheld device and out of nozzle 130, the air flow being capable of safely and effectively blowing the animal's coat to separate fur so that the user is able to examine the underlying skin for ailments or conditions. The user may also adjust the flow rate of air by actuating flow control 122 to select the desired flow rate. Buttons 124,139 may also be used to shine a light (e.g., one or more LEDs), a UV blacklight, or to take a photograph or video of a target area of interest. In some examples, any one, any combination or all of the buttons may be disposed on the base 160 instead of the tool 100. The handheld device may be moved over the entire body of the animal or only a portion until the examination is complete. Additionally, any data of interest (e.g., photographs, videos, etc.) may be stored in base 160 and/or transmitted wirelessly or through wired means to a computer or mobile device for additional processing, or to suggest an additional course of treatment, or for inclusion into a report on the animal's well-being for further veterinarian physical inspection.

In another example, diagnostic tool 500 of FIGS. 5A-C may include any of the features described above, but may include a physical magnifying lens 510 that is coupled to the diagnostic tool. Specifically, the housing of the diagnostic tool may have a groove 502 near the head region, and the magnifying lens 510 may have a ring 503 that fits within the groove 502 and is rotatable thereon so that lens 510 is capable of moving between a first open position (FIG. 5A) where it is positioned above the nozzle and in position for viewing through it by the user, and a second closed position (FIG. 5B) where it is positioned below the nozzle and away from the line of sight of the user. Magnifying lens 510 may be capable of magnifying the target area by 2×, 3× or 10×. A physical magnifying lens 510 may be used instead of a digital image capture and zoom, for example, to manufacture the device at a cheaper cost, or may be included in addition to the digital image capture and zoom feature.

Although the invention herein has been described with reference particular embodiments, it is be understood that these embodiments are merely illustrative of the principles and applications of the present invention. For example, the size, power and flow rate of the diagnostic tool may be scaled up or down for uses in wildlife or zoo applications. It is therefore to be understood that numerous modifications may be made to the illustrative embodiments and that other arrangements may be devised without departing from the spirit and scope of the present invention as defined by the appended claims.

It will be appreciated that the various dependent claims and the features set forth therein can be combined in different ways than presented initial claims. It will also be appreciated that the features described in connection with individual embodiments may be shared with others of the described embodiments.

We claim:

1. A handheld diagnostic device comprising:
a housing having a proximal end and a distal end;
a magnifying element coupled to the housing;
a nozzle;
at least one light; and
a flow control configured and arranged to adjust a flow rate of a gas flowing through the nozzle.

2. The handheld diagnostic device of claim 1, wherein the housing includes a body, a neck and a face.

3. The handheld diagnostic device of claim 2, wherein the nozzle is disposed on the face.

4. The handheld diagnostic device of claim 2, wherein the flow control includes a wheel or digital switch, and an on-and-off switch disposed on the body.

5. The handheld diagnostic device of claim 2, wherein the magnifying element includes a lens couple to the body and rotatable relative thereto and configured to transition between a first position above the nozzle and a second position below the nozzle.

6. The handheld diagnostic device of claim 2, wherein the magnifying element includes a camera to capture an image and transmit it to a display, and a zoom toggle disposed on the housing to zoom in and out.

7. The handheld diagnostic device of claim 2, further comprising at least blacklight disposed on the face.

8. The handheld diagnostic device of claim 2, further comprising a camera lens disposed on the face.

9. The handheld diagnostic device of claim 2, further comprising a laser disposed on the face.

10. The handheld diagnostic device of claim 2, further comprising at least one LED, at least one blacklight, and a camera lens.

11. The handheld diagnostic device of claim 10, further comprising a series of buttons configured and arranged to control the at least one LED, the at least one blacklight, and the camera lens.

12. A system comprising:
the handheld diagnostic device of claim 1;
a base unit having a housing, and an air compressor disposed within the housing; and
a flexible tubing coupled to the handheld diagnostic device and the base unit and configured to deliver a gas from the air compressor to the nozzle.

13. The system of claim 12, further comprising a power source coupled to the base unit.

14. The system of claim 13, wherein the power source is an electrical outlet.

15. The system of claim 13, wherein the power source is a battery.

16. A method of examining an animal comprising:
providing a handheld diagnostic device including a housing having a proximal end and a distal end, a nozzle, a magnifying element, at least one light, and a flow control configured and arranged to adjust a flow rate of a gas flowing through the nozzle; and
directing a stream of gas out of the nozzle onto a target area of the animal to blow and separate the fur to see the animal's skin underneath the fur.

17. The method of claim 16, wherein providing a handheld diagnostic device comprises providing a device having an LED.

18. The method of claim 16, wherein providing a handheld diagnostic device comprises providing a device having a camera.

19. The method of claim 18, further comprising the step of taking a photograph of the target area with the camera.

20. The method of claim 19, further comprising the step of taking a video of the target area with the camera.
